(19) United States
(12) Patent Application Publication
(10) Pub. No.: US 2026/0210926 A1
(43) Pub. Date: Jul. 23, 2026

(54) Systems and Methods for Low-Carbon Creation of Reactive Media

(71) Applicant: Thalo Labs, Inc., New York, NY (US)

(72) Inventors: Brendan Hermalyn, New York, NY (US); Julia Hestenes, New York, NY (US); Mihir Juvvadi, New York, NY (US); Samuel Patterson, New York, NY (US); Mellina Calzolaio, New York, NY (US); Nina Nappa, New York, NY (US); Brian Zimmerly, New York, NY (US); Peter Schultz, New York, NY (US)

(21) Appl. No.: 19/453,603

(22) Filed: Jan. 20, 2026

Related U.S. Application Data
(60) Provisional application No. 63/747,482, filed on Jan. 21, 2025.

(51) Int. Cl. G01N 33/00; B01D 53/62; B01D 53/81; C01F 11/06

(52) U.S. Cl. CPC G01N 33/004; C01F 11/06; G01N 33/0013; G01N 33/0016; B01D 53/62; B01D 53/81; B01D 2251/404; B01D 2251/602; B01D 2253/1124; B01D 2257/504

(57) ABSTRACT

An example system for creating a reactive media to capture a constituent gas from a gas mixture includes a housing into which a non-reactive media is positioned, a heating element for heating the non-reactive media in the housing which causes a release of gas and creates a reactive media that when in use is capable of capturing the constituent gas from inbound gas contacting the reactive media, an emissions measuring device including a plurality of sensors to detect concentrations of gas constituents and a gas flow sensor to detect a gas flow rate, and a computing device having one or more processors to perform functions including calculating an emission of a particular gas constituent in the gas vented (and captured to reduce or eliminate spread to the atmosphere) through the housing outlet based on a concentration of the particular gas constituent detected by the plurality of sensors and the gas flow rate detected by the gas flow sensor.

### CROSS REFERENCE TO RELATED APPLICATION

The present application claims priority to U.S. Application Number 63/747,482, filed on Jan. 21, 2025, the entire contents of which are herein incorporated by reference.

### FIELD

The disclosure relates generally to a system for creating a reactive media to capture a constituent gas (or stream of gas) from a source, and more particularly to, a new and useful system for measuring an amount of emission of a particular gas constituent during creation of the reactive media and a feedback loop for calculation of an overall carbon footprint.

### BACKGROUND

Carbon dioxide capture and storage processes CO2 over a range of concentrations, directly from outdoor or indoor air to directly emitting sources, such as fossil-fuel power plants, boilers, and cement plants. To do so, some methods include using a sorbent to separate carbon dioxide from a solution of gasses, and once absorbed by the sorbent, the CO2-sequestered material can be separately stored, disposed of, or used as needed.

Traditional methods of generation of a reactive media, such as those used as capture sorbents and cementitious materials, usually do not focus on any carbon impact during creation of the sorbent. Some methods use petrochemical ignition as heat sources (e.g., natural gas) to create the reactive media, and then normally vent both emissions from combustion and emissions from a decomposition process (e.g., converting CaCO3 to CaO+CO2) to the atmosphere, thereby contributing a significant amount of greenhouse gas emissions while creating a reactive media that will be used to capture such gasses. Other methods of reactive media generation, such as when generating reactive media in a form of quicklime, typically result in a release carbon dioxide as well. As a result, traditional methods of reactive media generation negate a lot of the benefits for which the sorbent is intended.

### SUMMARY

In one example, a system for creating a reactive media to capture a constituent gas from a gas mixture is described. The system comprises a housing into which a non-reactive media is positioned and the housing includes a housing outlet to vent the housing, and a heating element for heating the non-reactive media in the housing which causes a release of gas and creates a reactive media and the gas is vented through the housing outlet. The system also includes an emissions measuring device, including a plurality of sensors to detect concentrations of gas constituents and a gas flow sensor to detect a gas flow rate, coupled to the housing outlet, and a computing device having one or more processors, to perform functions comprising: calculating an emission of a particular gas constituent in the gas vented through the housing outlet based on a concentration of the particular gas constituent detected by the plurality of sensors and the gas flow rate detected by the gas flow sensor.

In another example, a system for creating a reactive media to a constituent gas from a gas mixture is described. The system comprises a housing into which a non-reactive media is positioned and the housing includes a housing outlet to vent the housing, and a heating element for heating the non-reactive media in the housing which causes a release of gas and creates a reactive media and the gas is vented through the housing outlet. The system also includes a gas capture device coupled to the housing outlet to receive the gas vented from the housing and to capture the gas vented from the housing. The system also includes an emissions measuring device, including a plurality of sensors to detect concentrations of gas constituents and a gas flow sensor to detect a gas flow rate, coupled to the housing outlet. The system also includes a computing device having one or more processors, to perform functions comprising: calculating an emission of a particular gas constituent in the gas vented through the housing outlet based on a concentration of the particular gas constituent detected by the plurality of sensors and the gas flow rate detected by the gas flow sensor, and calculating a carbon footprint metric of the reactive media based on an amount of the emission of the particular gas constituent released during creation of the reactive media and an amount of the constituent gas that the reactive media is capable of capturing.

In another example, a method for creating a reactive media to a constituent gas from a gas mixture is described. The method comprises heating a non-reactive media positioned in a housing which causes a release of gas and creates a reactive media and the gas is directed through a housing outlet. The method also comprises detecting, by a plurality of sensors, concentrations of gas constituents in the gas directed through the housing outlet, detecting, by a gas flow sensor, a gas flow rate of the gas directed through the housing outlet, and calculating, by a computing device having one or more processors, an emission of a particular gas constituent in the gas directed through the housing outlet based on a concentration of the particular gas constituent detected by the plurality of sensors and the gas flow rate detected by the gas flow sensor.

While examples describe use of the reactive media for capture of gases, such produced media may also be used for other relevant industrial applications, such as but not limited to, cementitious materials, flue gas treatment, chemical manufacturing, and soil stabilization.

The features, functions, and advantages that have been discussed can be achieved independently in various examples or may be combined in yet other examples. Further details of the examples can be seen with reference to the following description and drawings.

### BRIEF DESCRIPTION OF THE FIGURES

Examples, objectives and descriptions of the present disclosure will be readily understood by reference to the following detailed description of illustrative examples when read in conjunction with the accompanying drawings, wherein:

FIG. 1 illustrates a system for creating a reactive media to capture a constituent gas from a gas stream, according to an example implementation.

FIG. 2 illustrates another example of the system of FIG. 1 in which a gas capture device is included, according to an example implementation.

FIG. 3 is a high level process flow diagram using the system of FIG. 1 or FIG. 2 to create reactive media for use, according to an example implementation.

FIG. 4 is a block diagram illustrating components for implementing the high level process flow diagram of FIG. 3, according to an example implementation.

FIG. 5 is another high-level process flow diagram using the system of FIG. 1 or FIG. 2 to create reactive media for another use, according to an example implementation.

FIG. 6 is a flowchart illustrating an example of a method for creating a reactive media to a constituent gas from a gas stream, according to an example implementation.

FIG. 7 illustrates a block diagram of a computing device, according to an example implementation.

### DESCRIPTION OF THE EMBODIMENTS

Disclosed examples will now be described more fully hereinafter with reference to the accompanying drawings. Several different examples are described and should not be construed as limited to all possible alternatives. Rather, these examples are described so that this disclosure is thorough and complete and fully conveys a scope of the disclosure to those skilled in the art.

For oxide based carbon capture systems, a main barrier to fully carbon negative lifecycle systems lies not in the capture of carbon nor in the final sequestration. Instead, a majority of emissions related to direct mineralization techniques involves generation of reactive material itself that is used to capture the carbon. Traditional methods of generation of reactive media for carbon capture or cementitious applications usually have not focused on the carbon impact and have used petrochemical combustion as heat sources (e.g., natural gas) and have normally vented both emissions from combustion and emissions from a decomposition process (e.g., converting CaCO3 to CaO+CO2) to the atmosphere, thereby contributing a significant amount of greenhouse gas emissions.

Within examples described herein, systems and methods are described to create this same reactive mineral (such as CaO or Ca(OH)₂) in a low-or-zero carbon fashion. To do so, renewable energy sources (e.g., electricity) are preferred for powering heating devices and all stages of the system. An amount of emissions created during generation of the reactive media is monitored and tracked as a verification that a net zero or net negative carbon footprint results once the created reactive media is used to capture carbon dioxide. In addition, an amount of total power used, as a function of time, is measured to be able to calculate total emissions of use, for example, as a function of time to enable optimization at different times of day (more generation at night during off peak and lower emission power or matching to demand curves for similar reasons).

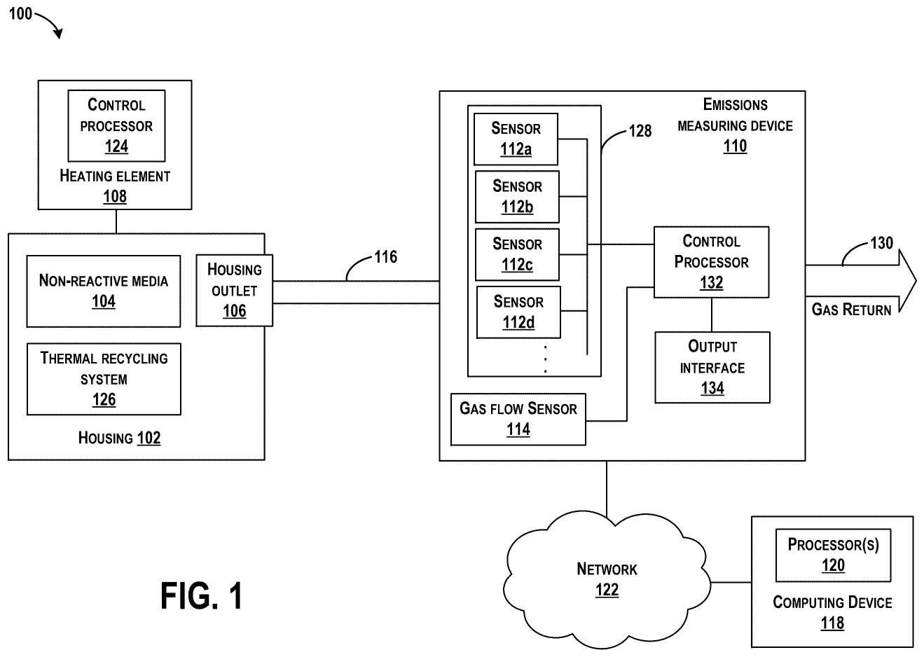

Referring now to the figures, FIG. 1 illustrates a system 100 for creating a reactive media to capture a constituent gas from a source (e.g., a gas mixture) or for use in an industrial application (e.g., cementitious materials, chemical manufacturing, flue gas treatment, or soil stabilization), according to an example implementation. The system 100 is operated to create the reactive media, and then calculate how much carbon dioxide (or other constituent gas) is generated during creation of the reactive media as a basis to calculate an overall negative carbon footprint estimation that can be achieved with the reactive media used to capture carbon dioxide or for a carbon-benefit industrial application, for example. The reactive media created can then be used separately to achieve the environmental benefits, such as to capture constituent gasses from a gas mixture in any number of use cases.

The system 100 includes a housing 102 into which a non-reactive media 104 is positioned, and the housing 102 includes a housing outlet 106 to vent the housing 102. The system 100 also includes a heating element 108 for heating the non-reactive media 104 in the housing 102 which causes a release of gas and creates a reactive media, and the gas released is vented through the housing outlet 106. The system 100 also includes an emissions measuring device 110, including a plurality of sensors 112a-d to detect concentrations of gas constituents and a gas flow sensor 114 to detect a gas flow rate, coupled to the housing outlet 106 such as via an exhaust pathway 116. The system 100 further includes a computing device 118 having one or more processors 120, to perform functions including calculating an emission of a particular gas constituent in the gas vented through the housing outlet 106 based on a concentration of the particular gas constituent detected by the plurality of sensors 112a-d and the gas flow rate detected by the gas flow sensor 114.

As used herein, gas “vented” through an outlet refers to gas being directed through the outlet and does not require release to the atmosphere. In various embodiments, the gas is directed to capture, vent or purge, gas recycle, compression, utilization, or sequestration processes.

FIG. 1 illustrates the computing device 118 in communication with the emissions measuring device 110 via a network 122. The network 122 can include a local area network (LAN), a wide area network (WAN), or the Internet. In other examples, communications between the emissions measuring device 110 and the computing device 118 can be direct via wired or wireless communications.

The housing 102 is a sealed housing, in one example, and includes a platform or other holder into which the non-reactive media 104 is positioned. For example, the housing 102 is a vacuum sealing housing.

The non-reactive media 104 can take many forms or include many different materials or combinations of materials. In one example, the non-reactive media 104 is a carbonate. In another example, the non-reactive media 104 is a carbonate input. In still further examples, the non-reactive media 104 includes carbonates such as magnesium, calcium, potassium, lithium, etc., or similar. Still further, additives or doping agents are added to the non-reactive media 104, in some examples, to increase reactivity or a rate of reactivity. Thus, the non-reactive media 104 may be a virgin, unprocessed single material, mix of materials, or a majority single material with small quantities of additives defined for purposes of increasing rate of carbon intake. Based on a type of material of the non-reactive media, different types of reactive media are generated for capturing a particular constituent gas or mix of gasses.

In further examples, the non-reactive media 104 includes precursors for battery materials synthesis. For instance, the non-reactive media 104 may include one or more transition metal carbonate precursors and a lithium or sodium source (e.g., lithium or sodium hydroxide and/or lithium or sodium carbonate), and heating causes evolution of CO₂ and formation of lithium or sodium transition metal oxides. The emissions measurement, carbon accounting, and control techniques described herein apply similarly to such battery materials synthesis processes.

The heating element 108 is shown separate from the housing 102 in FIG. 1. In this example, the heating element 108 provides heat into the housing 102 through external conduction. In other examples, the heating element 108 is included inside the housing 102 for direct heating of an internal portion of the housing including the non-reactive media 104. Notably, the heating element 108 is positioned in close physical proximity to the housing 102 (whether inside the housing 102 or external to the housing 102) so as to minimize loss of heat during transmission. An example of physical proximity of the heating element 108 to the housing 102 includes about 3-5 feet (more or less due to tolerances of positioning that can include 1-2 feet).

The heating element 108 heats the non-reactive media 104 (e.g., carbonate input) causing a release of carbon dioxide (e.g., gas) and creating a reactive media (e.g., metal oxide). The carbon dioxide is vented through the housing outlet 106.

The heating element 108 can take many forms. The heating element 108 includes resistive elements, such as within the housing 102 and the heating element 108 comprises resistive elements of an electric kiln or rotary furnace, in one example. In another example, the heating element 108 uses one or more of direct microwave heating, thermal focusing, heating from regeneration of a secondary process, and heating through plasma processes. Within examples, the heating element 108 is powered by a source of power including electric power, hydroelectric power, nuclear power, or a renewable power source.

The heating element 108 is shown to include a control processor 124 to control operation of the heating element 108, through use of a source of power, based on a temporal or weather based parameter including a time of day. It is desirable to use electric power, either sourced from a clean grid area (such as one with a direct feed from a hydroelectric power, nuclear power, or renewable power such as solar or wind) or from an onsite green power source (including, but not limited to, solar, wind, hydroelectric, or some combination possibly with battery power). In some instances, one or more of these sources of power can become unavailable due to weather, time of day, etc., and thus, the control processor 124 monitors availability and directs power from a selected source accordingly. To do so, the control processor 124 can access sources of power (or controllers of the sources of power) via the network 122, and thus is able to communicate via the network 122 with all available sources of power. When a temporal or weather-based component, such as night time for solar power sources or low wind for wind-turbine power sources, is present, the control processor 124 selects a next available power source having a low or zero carbon output.

The heat inside the housing 102 is required for generation of the reactive media by heating the non-reactive media 104 according to a heat cycle. An example heat cycle, such as for metal carbonate or other carbonates, includes temperature of decomposition of about 850 degrees C. A residence time depends on physical parameters of heat dispersion in the system.

A resultant reactive media that is generated is capable of capturing a constituent gas from inbound gas contacting the reactive media, for example. In some examples, the reactive media is positioned such that inbound gas passes through the reactive media. In any configuration, the reactive media can react with the constituent gas in order to remove the constituent gas from the inbound gas, for example. Still other example uses exist for the resultant reactive media that is generated including, but not limited to, cementitious materials, chemical manufacturing, flue gas treatment, and soil stabilization. As used herein, “reactive media” includes, in various embodiments, metal oxides and/or hydroxides, such as mixed oxides, doped compositions, and blends, and may be provided in any suitable physical form including powders, granules, pellets, or briquettes. In some embodiments, the reactive media is reactive chemically, catalytically, and/or electrochemically.

The housing 102 is also shown to include a thermal recycling system 126 within the housing 102 for receiving and maintaining heat from the heating element 108 and for broadcasting maintained heat in the housing 102 enabling non-continuous operation of the heating element 108 while still maintaining a desired temperature inside the housing 102 capable of creating the reactive media. The thermal recycling system 126 can take a number of forms, such as a routing system to route an input of a rotary kiln over exhaust of the non-reactive media 104 to pre-heat the non-reactive media 104. In another example, the thermal recycling system 126 includes a thermal mass buffer to maintain heat inside the housing 102. Thus, the thermal recycling system 126 can simulate operation of the heating element 108 (or enabling heat recovery) by maintaining a temperature inside the housing 102 at a temperature of decomposition needed for the non-reactive media. Use of the thermal recycling system 126 is optional, and may be selectively enabled by the control processor 124 of the heating element 108. For example, the thermal recycling system 126 can be a passive device that can be automatically (or manually) inserted into or removed from the housing 102 based on outputs of the control processor 124. In another example, the thermal recycling system 126 is an active device (separate smaller heat source) that is activated by outputs of the control processor 124. By doing so, the heating element 108 can be turned off or operated intermittently to reduce consumption of power and reduce any creation of carbon emissions by the power sources. Intermittent operation of the heating element 108 can be based on power grid demand, a requirement to satisfy base demand of renewable power sources, or other factors.

The emissions measuring device 110, including a plurality of sensors 112a-d, are in a pathway of the gas exiting the housing outlet 106 to detect concentrations of particular gas constituents. The exhaust pathway 116 includes plastic tubes or flexible hoses, for example, which fluidly couple gas exiting the housing outlet 106 with the sensors 112a-d and the gas flow sensor 114, respectively. The emissions measuring device 110 optionally includes a pump to pull a side-stream of air (e.g., about 1 liter/min) from the housing outlet 106, for example.

In one example, a gas processing device is coupled to or in the exhaust pathway 116 to perform cooling of the gas, filtering of any particles, or trapping of moisture within the gas prior to gas entering the emissions measuring device 110 and contacting sensors.

The emissions measuring device 110 can include a watertight sealed housing, and the sensors 112a-d are arranged in a sensor manifold 128. The sensors 112a-d are thus positioned in close proximity to minimize a volume of gas needed for processing. A reduced gas volume also minimizes a response time of the sensors 112a-d from any event that causes a change in composition of the gas stream. Although four sensors are shown in FIG. 1, more or fewer sensors can be included. In addition, in other examples, a single sensor capable of detecting concentrations of multiple different types of gasses is used.

In examples, the emissions monitoring device 110 samples from received gas to pass samples to the sensors 112a-d for processing. In other examples, the emissions monitoring device 110 is a flow-through, on-line monitoring device at the exhaust pathway 116 such that all exhausted gas flows through and is constantly monitored rather than sampling. The emissions monitoring device 110 thus operates to sample gas intermittently or to process gas on a continuous gas sensing basis.

Following processing of the gas by the sensors 112a-d, the gas is provided through a gas return pathway 130 back to an exhaust stack.

The sensors 112a-d detect concentrations of a set of greenhouse gas constituents, and the sensors 112a-d take the form of gas sensors to detect concentrations of gas constituents including carbon dioxide, formaldehyde (e.g., product of incomplete combustion of a fuel source), carbon monoxide, particulate matter (e.g., a product of burning coal or diesel fuel), sulfur oxide, nitrogen oxide, methane, and oxygen. Thus, the sensors 112a-d may include any of CO2 sensors, CH4 sensors, HCHO sensor, CO sensor, O2 sensor, NO2 sensor, SO2 sensor, and a pressure-humidity-temperature (PHT) sensor, for example. In other examples, the sensors 112a-d also include one or more of a temperature sensor, a humidity sensor, a pressure sensor, and additionally or alternatively a geospatial location sensor. In yet other examples, the sensors 112a-d include a spectrometer to detect the concentrations of the gas constituents in the gas based on an intensity of detected light.

Within examples, the gas flow sensor 114 takes a form of one of a wire anemometer, a pitot tube, or other gas flux sensor configured to track gas flow rate through the exhaust pathway 116.

The emissions measuring device 110 also includes a control processor 132, having one or more processors, to sample outputs of the sensors 112a-d and the gas flow sensor 114, such as at a rate of once per minute or once per hour, and store received data in local memory. In one example, the emissions measuring device 110 includes an output interface 134, which can be a wired or wireless communication interface (e.g., wireless transmitter and receiver) to stream received data to the computing device 118.

Thus, the control processor 132 samples the sensors 112a-d and the gas flow sensor 114 continuously or intermittently, compresses and/or stores raw sampled data locally, and intermittently broadcasts the raw data to the computing device 118, such as once per day. In an alternate example, the control processor 132 locally processes raw sampled data, and then transmits derived data to the computing device 118, such as once per day (rather than transmitting all sampled data).

The system 100 in FIG. 1 illustrates the computing device 118 having one or more processors 120 separate from the emissions measuring device 110. In other examples, the computing device 118 is arranged in the emissions measuring device 110 and may be combined with (or separate from) the control processor 132.

The computing device 118 calculates an emission of a particular gas constituent in the gas vented through the housing outlet 106 based on a concentration of the particular gas constituent detected by the plurality of sensors 112a-d and the gas flow rate detected by the gas flow sensor 114. For example, the computing device 118 performs functions of calculating a total emissions of the particular gas constituent being tracked during a time period by integrating a concentration of the particular gas constituent detected by the plurality of sensors 112a-d, multiplied by the gas flow rate detected by the gas flow sensor 114, over the time period. The computing device 118 also can calculate an emission rate of the particular gas constituent being tracking during the time period by dividing the total emissions by a duration of the time period, for example.

Within examples, the computing device 118 tracks the emission of a particular gas constituent in the gas vented through the housing outlet 106, during creation of the reactive media, in order to determine a carbon footprint of the reactive media created by the system. As one example, the computing device 118 determines an amount of power utilized by the heating element 108 that is based on a source of power including a fossil fuel, and calculates a carbon footprint metric of the reactive media based on (i) the amount of power utilized by the heating element 108 that is based on the source of power including the fossil fuel, (ii) an amount of the emission of the particular gas constituent released during creation of the reactive media, and (iii) an amount of the constituent gas that the reactive media is capable of capturing. For example, an amount of power utilized maps to an amount of carbon emissions (based on the type of power source or based on power consumption measured at the heating element 108 by a measurement device and output to the computing device 118), which can be added to the amount of emission of the gas constituent measured during creation of the reactive media for a total emissions that are subtracted from an amount of the constituent gas that the reactive media is capable of capturing to generate the carbon footprint metric of the reactive media. Generation of the reactive media may result in 200 kg of emissions, but if such reactive media is capable of capturing 1 ton of emissions, the process has a net negative carbon footprint.

In similar examples, the computing device 118 additionally determines a power source carbon footprint based on power utilized by the heating element from a source of power, and calculates a carbon footprint metric of the reactive media based on (i) the power source carbon footprint, (ii) an amount of the emission of the particular gas constituent released during creation of the reactive media, and (iii) an amount of the constituent gas that the reactive media is capable of capturing. The power source carbon footprint may additionally be based on power used by an entirety of the system 100, which can include further components such as compressors, etc. The power source carbon footprint is based on an amount of power utilized and can be information received from the power sources via the network 122.

In still further examples, the computing device 118 receives an input indicating an amount of the reactive media created, determines based on the amount of the reactive media created an amount of the constituent gas that the reactive media is capable of capturing, and calculates a carbon footprint metric of the reactive media based on an amount of the emission of the particular gas constituent released during creation of the reactive media and an amount of the constituent gas that the reactive media is capable of capturing. Thus, a carbon footprint metric can be related to an overall output of the system 100 as well. In this example, the computing device 118 can be programmed to modify one or more parameters of the system 100 based on the carbon footprint metric being above a threshold. For instance, a source of power for the heating element 108 can be changed, such as if the system 100 was using natural gas (due to costs from different power sources, lack of solar power, etc.), the computing device 118 can cause the system 100 to switch to a renewable energy power source. In another example, the heating element 108 can be operated intermittently by relying on heat from the thermal recycling system 126 instead of the heating element 108 (which may cause longer generation times), or other parameters as well can be changed. In still another example, the computing device 118 pauses operation of the system 100 until renewable power sources are available to operate the system 100 more efficiently.

In further examples, the computing device 118 is configured to modify one or more process parameters based on the measured outlet gas composition (e.g., CO₂ and/or other constituents) and measured outlet gas flow rate, including but not limited to, material throughput rate, temperature or heat input, kiln or furnace rotation rate, and process-gas feedthrough rate (e.g., air, N₂, O₂).

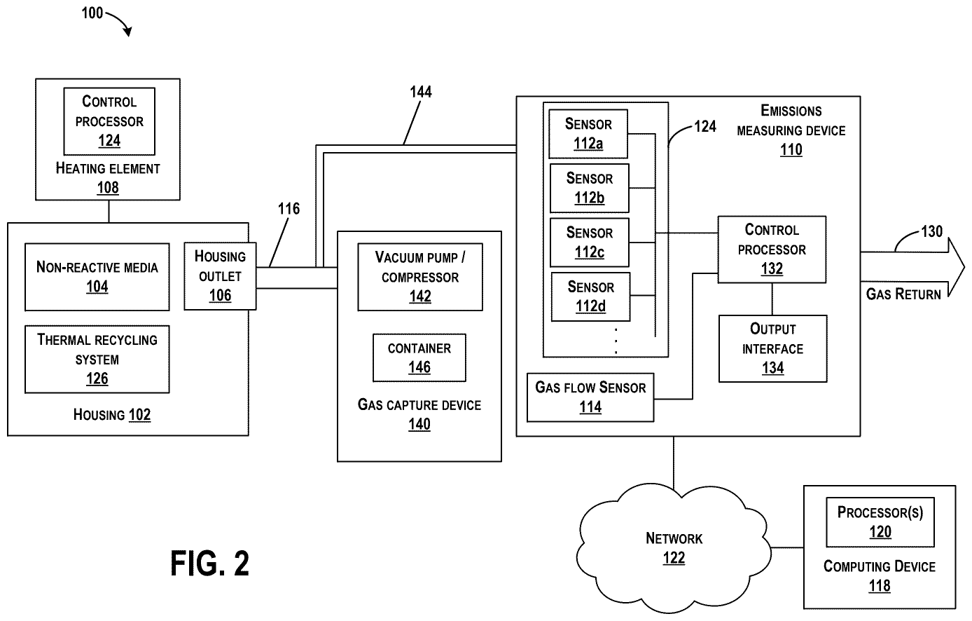

FIG. 2 illustrates another example of the system 100 of FIG. 1 in which a gas capture device 140 is included, according to an example implementation. The gas capture device 140 is coupled to the housing outlet 106 to receive the gas vented from the housing outlet 106 and to capture the gas vented from the housing outlet 106. The gas capture device 140 includes a vacuum pump or compressor 142 to actively pull processed gas out of the housing 102 and into the gas capture device 140, in one example.

The emissions measuring device 110 also monitors the gas vented from the housing outlet 106 by coupling to the exhaust pathway 116 via another exhaust pathway 144, so as to tap into the exhaust pathway 116. In this example, the emissions measuring device 110 monitors the free stream carbon dioxide released during creation of the reactive media prior to capture by the gas capture device 140 to determine amounts of carbon dioxide released during generation of the reaction media and to determine amounts of carbon dioxide captured.

In one example, the gas capture device 140 compresses the gas constituent for storage in a container 146. The container 146 is shown inside the gas capture device 140 in FIG. 2. In other examples, the container 146 is separate from the gas capture device 140, or at least removable from the gas capture device 140.

The arrangement of the system 100 shown in FIG. 2 may further improve operations of the system 100 toward an overall negative carbon footprint creation of the reactive media. For example, the gas capture device 140 is operated to capture a vented gas (i.e., carbon dioxide) that is released during creation of the reactive media, and the computing device 118 then calculates an emission of a particular gas constituent in the vented gas based on a concentration of the particular gas constituent detected by the plurality of sensors 112a-d and the gas flow rate detected by the gas flow sensor 114 from gas received via the exhaust pathway 144. The computing device 118 calculates a carbon footprint metric of the reactive media based on an amount of the emission of the particular gas constituent released during creation of the reactive media and an amount of the constituent gas that the reactive media is capable of capturing. In addition, in examples where all gas released during generation is captured, the carbon footprint metric of the reactive media improves.

The system 100 can be configured to include multiple gas capture devices, and the computing device 118 can be configured to be in communication with the gas capture devices (either via a direct wired or wireless communication, or via the network 122). In one example, the computing device 118 controls operation of the gas capture devices to include more or fewer online based on outputs of the emissions measuring device 110. If the concentration of the particular gas constituent detected by the plurality of sensors 112a-d is high, the computing device 118 causes one or more additional gas capture devices to be online for further filtering of gas exhausts of the housing 102. In other examples, if the concentration of the particular gas constituent detected by the plurality of sensors 112a-d is low, the computing device 118 causes one or more gas capture devices to be offline to reduce filtering of gas exhausts of the housing 102 and operate the system 100 more efficiently. Each gas capture device can be connected to the exhaust pathway 116 via valves operable to be open and shut via commands received from the computing device 118, for example.

In another example, the gas capture device 140 includes a compression system (e.g., series of compressors) to capture CO2 that is released during the reactive media generation process. Once captured, the CO2 can be disposed of underground or disposed in a class VI well, the CO2 can be added to media like concrete for permanent sequestration, the CO2 can be chemically or electrochemically (catalysis) converted into a different valuable material such as synthetic aviation fuel, or the CO2 can be converted or destroyed into a material with a lower greenhouse gas (GHG) potential such as carbon monoxide or elemental carbon.

The system 100 (shown in FIG. 1 or 2) is operable to estimate a complete picture of a carbon footprint for generation of the reactive media. The computing device 118 calculates an emission of a gas constituent (either in gas output from the housing 102 via the exhaust pathway 116 or the exhaust pathway 144) based on a concentration of the particular gas constituent detected by the plurality of sensors 112a-d and the gas flow rate detected by the gas flow sensor 114, and determines a power source carbon footprint based on power utilized by the heating element 108 from the power source, and calculates a carbon footprint metric of the reactive media based on (i) the power source carbon footprint, (ii) an amount of the emission of the particular gas constituent released during creation of the reactive media, and (iii) an amount of the constituent gas that the reactive media is capable of capturing.

The system 100 as a whole is operated to create the reactive media (for separate use to capture carbon emissions) with a net zero or net negative carbon footprint (without having to buy carbon credits). Measurements of gas emissions are performed during creation of the reactive media to enable a verification that an amount of carbon generated (during creation) is less than an amount of carbon that is capable of being captured by the reactive media that is generated.

By offering measurements during generation of the reactive media, a verified process can be provided to know how much carbon emissions were produced to generate reactive media, which itself is capable of capturing around 40% of its weight in CO2, for example. Reducing the prevalence of carbon dioxide is essential, but if more carbon dioxide was generated during creation of the media used to capture carbon dioxide, then there is no net benefit. The system 100 offers a closed loop analysis benefit with measurements, which can be used in real-time during creation of the reactive media to modify parameters of the system 100 accordingly and to control creation in a way that guarantees an overall net negative carbon footprint.

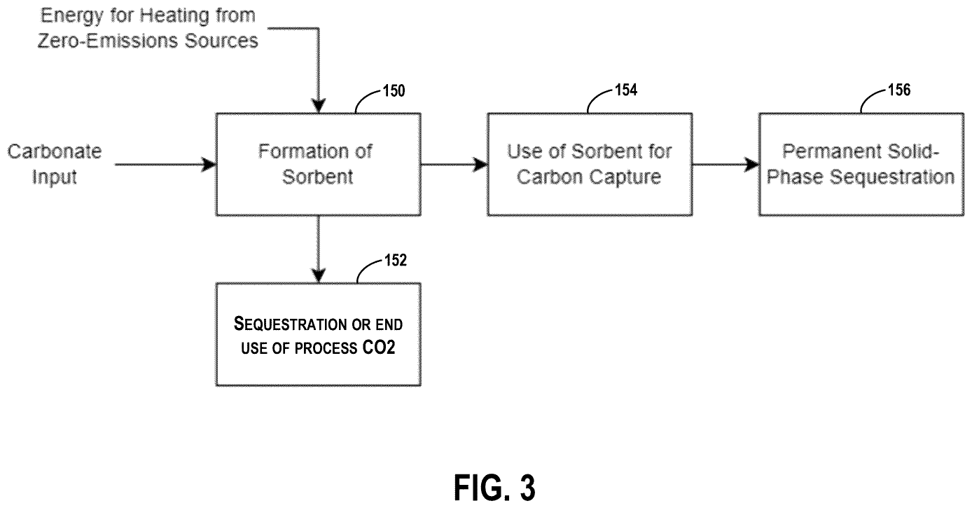

FIG. 3 is a high-level process flow diagram using the system 100 of FIG. 1 or FIG. 2 to create reactive media for use, according to an example implementation. In FIG. 3, the reactive media is described as a sorbent for use as a carbon capture material, however, the reactive media is usable for other industrial applications as described herein. Thus, the example reactive media generation processes described herein are applicable to generate reactive media for many uses and can be tailored in specific ways to generate a specific kind or type of reactive media.

At block 150, formation of a sorbent (e.g., a reactive media) occurs with inputs of the carbonate input and energy for heating from zero-emissions sources (preferably) such as renewable energy. A sorbent refers to a chemical or material used in an absorption of another substance. In the context of this description, sorbent refers to a chemical to absorb or capture a particular constituent gas, such as carbon dioxide, from air streams.

At block 152, a sequestration or end use of process carbon dioxide generated during formation of the sorbent occurs. For example, carbon dioxide (or other greenhouse gases) released during decomposition of the carbonate input can be prevented from entering the atmosphere through a variety of methods, including: carbon dioxide compression and/or liquefaction with transport via vehicle or pipeline to sequestration or re-use in other materials, direct transformation via catalysis, electrolysis, or another chemical process, dissociation to elemental materials through plasma or other methods, or through direct capture by a capture device (e.g., as shown in FIG. 2 by the gas capture device 140).

Once the sorbent is generated, the sorbent can be used in many different applications for carbon capture. Thus, at block 154, use of the sorbent for carbon capture is shown, such as within gas capture devices positioned proximal to gas exhausts of buildings, for example. The generation of the sorbent occurs first, followed by use of the sorbent in a capture phase. Note that described above, any CO2 generated during formation of the sorbent can also be captured/compressed, etc. and that is separate from use of the generated sorbent to capture CO2. The generation of sorbent does not use sorbent to capture the CO2 that comes out during generation. Any CO2 that comes out during generation of the sorbent is relatively high purity CO2 and can generally be compressed rather than requiring use of sorbent for capture.

At block 156, permanent solid-phase sequestration is performed of the used sorbent material that has captured carbon dioxide.

Note that the process described in FIG. 3 can be used to generate other low carbon materials for industries other than carbon capture uses, such as concrete and aggregate industries.

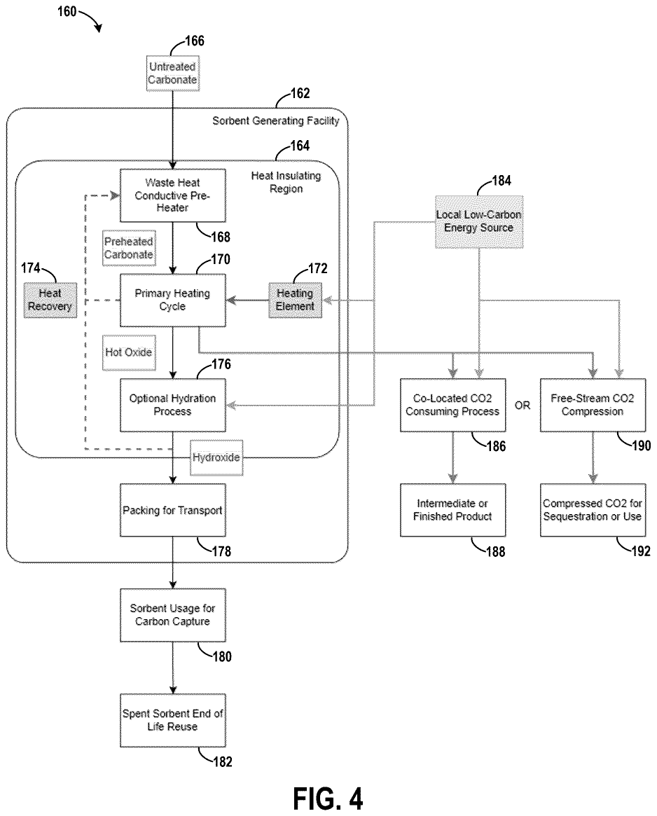

FIG. 4 is a block diagram illustrating components for implementing the high level process flow diagram of FIG. 3, according to an example implementation. A system 160 is illustrated including a sorbent generating facility 162 that includes a heat insulating region 164 for heating untreated carbonate 166 to create reactive media. The facility 162 and/or the heat insulating region 164 may be the same as or similar to the housing 102 in FIG. 1 and FIG. 2.

Inside the heat insulating region 164, initially, a waste heat conducive pre-heater 168 applies low-potential heat (e.g., such as waste heat from another process, solar heat, or other) prior to calcination to reduce residence time and energy consumption of a future calcination step. A primary heating cycle 170, utilizing a heating element 172 and/or heat recovery mechanisms 174 (e.g., thermal recycling system shown in FIG. 1 and FIG. 2) is applied to the preheated carbonate to generate a hot oxide. Following, an optional hydration process 176 is applied since hydroxides, such as Ca(OH)₂ (instead of CaO), are relatively more reactive with CO2 in low temperature/pressure conditions (like ambient air).

In some examples, after the optional hydration process 176, a further optional mechanical process is performed to shape the material into a final form, such as granulation, extrusion, grinding so that the produced material can be provided in a powder form, pellet form, or any desired form factor depending on mode of use.

After completion, the sorbent is generated and packaged for transport at block 178. At block 180, the sorbent is then used for carbon capture at a destination, and at block 182 spent sorbent end of life reuse is performed. In some examples, applications for the end of life reuse include, regeneration into a reactive media by calcination or integration into industrial or consumer products such as supplementary cementitious materials, concrete aggregate materials, plastics, soaps, etc.

FIG. 4 illustrates other external components too including a local low-carbon energy source 184 supplying energy to the heating element 172 and optional hydration process 176. Outputs or emissions of carbon dioxide can be captured by a co-located carbon dioxide consuming process (block 186), which creates an intermediate or finished product of stored carbon dioxide (block 188). Additionally or alternatively, free-stream carbon dioxide compression can be used (block 190) followed by creation of compressed carbon dioxide for sequestration or use (block 192).

In further examples, all aspects of the system 160 shown in FIG. 4 are tracked and monitored for generation of carbon emissions in order to guarantee and verify that a net negative carbon footprint exists due to generation of the sorbent (which will capture more carbon emissions than generated by the system 160). This can include monitoring packaging and transport as well, to minimize operational complexity and maximize sorbent efficacy by packing into a configuration that may directly interface with capture systems (that are deployed relatively nearby).

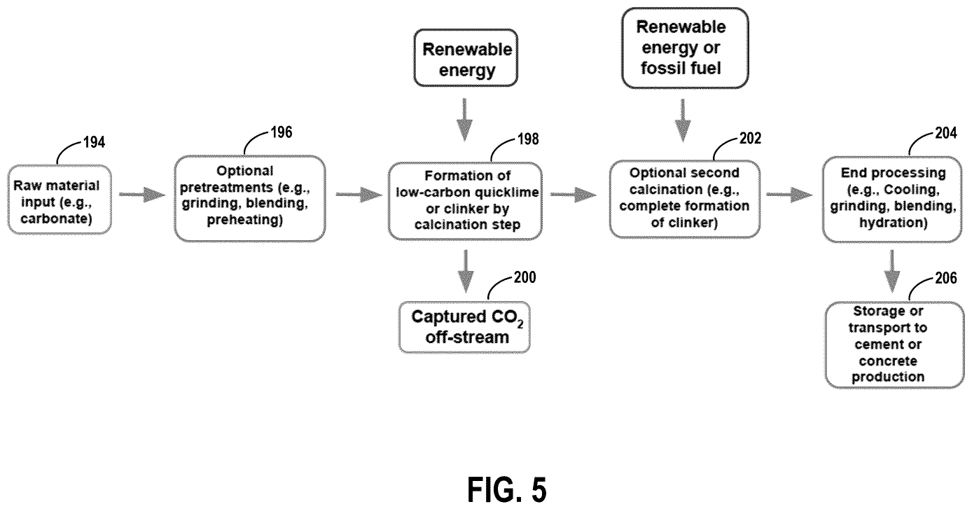

FIG. 5 is another high-level process flow diagram using the system 100 of FIG. 1 or FIG. 2 to create reactive media for another use, according to an example implementation. In the example shown in FIG. 5, an application for use of the generated reactive media includes low-carbon cement production. For comparison, examples in FIG. 3 and FIG. 4 refer to a carbon capture sorbent (which may be calcium oxide/hydroxide), and the example in FIG. 5 illustrates that a reactive media generation process can generate low-carbon quicklime (e.g., calcium oxide) and later calcium hydroxide or clinker for cementitious/concrete applications.

In FIG. 5, at block 194, a raw material input (e.g., carbonate) is provided, and at block 196, optional pre-treatments are applied (e.g., grinding, blending, preheating). At block 198, the plant directly generates low-carbon clinker (e.g., cement) from the carbonate in a single calcination step (such as at 1400° C.) while capturing CO2 and being powered by renewable energy, or the quicklime intermediate is first generated while capturing CO2 in a first calcination (such as at about 1000° C.) and powered by renewable energy and this material is then either 1) calcined a second time in a higher temperature kiln (e.g., at 1400° C.) to make clinker (fossil fuels may be required to reach this temperature) as shown at block 202, 2) is sent to another party to complete the clinker production at another facility, or 3) is hydrated to make calcium hydroxide which is used in various concrete applications. At block 200, any CO2 is captured. At block 204, processing ends with optional steps for cooling, grinding, blending, or hydration. At block 206, the generated reactive media is stored, or transported to a cement or concrete production facility.

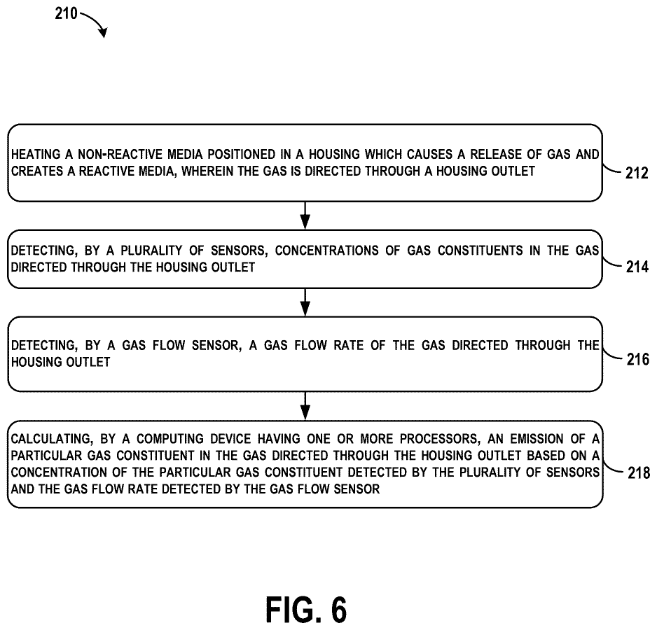

FIG. 6 is a flowchart illustrating an example of a method 200 for creating a reactive media to a constituent gas from a gas stream, according to an example implementation. Method 210 shown in FIG. 6 presents an example of a method that could be used with or performed by the system 100 in FIG. 1 and FIG. 2, the computing device 118 shown in the Figures herein, and the system 160 shown in FIG. 4, for example.

Within examples, devices or systems described herein are used or configured to perform logical functions presented in FIG. 6. In some instances, components of the devices and/or systems are configured to perform the functions such that the components are actually configured and structured (with hardware and/or software) to enable such performance. In other examples, components of the devices and/or systems are arranged to be adapted to, capable of, or suited for performing the functions, such as when operated in a specific manner. Method 210 includes one or more operations, functions, or actions as illustrated by one or more of blocks 212-218. Although the blocks are illustrated in a sequential order, these blocks may also be performed in parallel, and/or in a different order than those described herein. In addition, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, some blocks or portions of blocks may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium includes non-transitory computer readable medium or memory, for example, such as computer-readable media that stores data for short periods of time like register memory, processor cache and Random Access Memory (RAM). The computer readable medium additionally or alternatively includes non-transitory media, such as secondary or persistent long-term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 6, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 212, the method 210 includes heating a non-reactive media positioned in a housing which causes a release of gas and creates a reactive media, and the gas is directed through a housing outlet.

At block 214, the method 210 includes detecting, by a plurality of sensors, concentrations of gas constituents in the gas directed through the housing outlet.

At block 216, the method 210 includes detecting, by a gas flow sensor, a gas flow rate of the gas directed through the housing outlet.

At block 218, the method 210 includes calculating, by a computing device having one or more processors, an emission of a particular gas constituent in the gas directed through the housing outlet based on a concentration of the particular gas constituent detected by the plurality of sensors and the gas flow rate detected by the gas flow sensor.

In some examples, the method 210 additionally includes determining or directly measuring an amount of power utilized during the heating that is based on a source of power including a fossil fuel, and calculating, by the computing device, a carbon footprint metric of the reactive media based on (i) the amount of power utilized by the heating element that is based on the source of power including the fossil fuel, (ii) an amount of the emission of the particular gas constituent released during creation of the reactive media, and (iii) an amount of the constituent gas that the reactive media is capable of capturing. The carbon footprint metric is indicative of a sum of emissions resulting from every stage of generation of the reactive media, and a subtraction of emissions that the reactive media is capable of capturing.

In some examples, the method 210 additionally includes receiving an input indicating an amount of the reactive media created, determining, based on the amount of the reactive media created, an amount of the constituent gas that the reactive media is capable of capturing, and calculating, by the computing device, a carbon footprint metric of the reactive media based on an amount of the emission of the particular gas constituent released during creation of the reactive media and an amount of the constituent gas that the reactive media is capable of capturing.

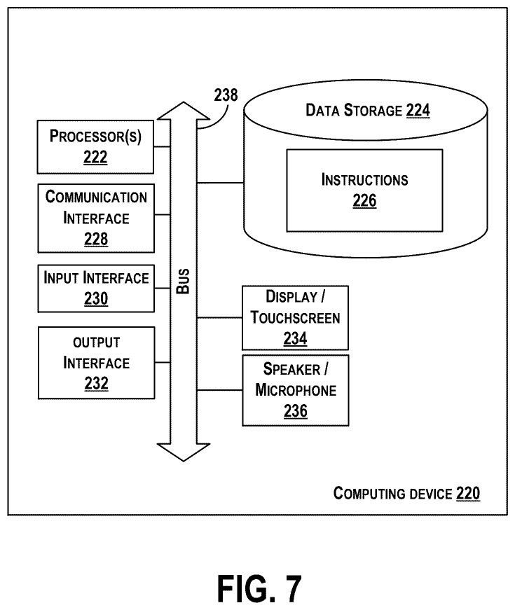

FIG. 7 illustrates a block diagram of a computing device 220, according to an example implementation. FIG. 7 does not necessarily show all of the hardware and software modules included in the computing device 220, and omits physical and logical connections that will be apparent to one of ordinary skill in the art after review of the present disclosure.

The computing device 220 in FIG. 7 is representative of any of the computing devices, control processors, or modules as described herein (including the computing device 118 shown in FIG. 1 and FIG. 2, for example).

The computing device 220 includes one or more processor(s) 222, and a non-transitory computer-readable media (data storage) 224 storing instructions 226, which when executed by the one or more processor(s) 222, causes the computing device 220 to perform functions (e.g., such as described in the flowchart of FIG. 6). To perform functions, the computing device 220 includes a communication interface 228, an input interface 230, an output interface 232, and optionally includes a display/touchscreen 234 and a speaker/microphone 236, and each component of the computing device 220 is connected to a communication bus 238. The computing device 220 may also include hardware to enable communication within the computing device 220 and between the computing device 220 and other devices (not shown). The hardware may include transmitters, receivers, and antennas, for example.

The communication interface 228 is a wireless interface and/or one or more wireline interfaces that allow for both short-range communication and long-range communication to one or more networks or to one or more remote devices. Such wireless interfaces provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 228 is configured to receive input data from one or more devices, and configured to send output data to other devices.

The data storage 224 includes or takes the form of memory, such as one or more computer-readable storage media that can be read or accessed by the one or more processor(s) 222. The computer-readable storage media includes volatile and/or non-volatile storage components, such as optical, magnetic, organic or other memory or disc storage, which can be integrated in whole or in part with the one or more processor(s) 222. The non-transitory data storage 224 is considered non-transitory computer readable media. In some examples, the non-transitory data storage 224 is implemented using a single physical device (e.g., one optical, magnetic, organic or other memory or disc storage unit), while in other examples, the non-transitory data storage 224 is implemented using two or more physical devices. The non-transitory data storage 224 thus is a computer readable medium, and instructions 164 are stored thereon. The instructions 226 include computer executable code.

The one or more processor(s) 222 include a general-purpose processor or special purpose processor (e.g., digital signal processors, application specific integrated circuits, etc.). The one or more processor(s) 222 receives inputs from the communication interface 228 as well as from other components (e.g., the display/touchscreen 234 or the speaker/microphone 236), and processes the inputs to generate outputs that are stored in the non-transitory data storage 224. The one or more processor(s) 222 are configured to execute the instructions 226 (e.g., computer-readable program instructions) that are stored in the non-transitory data storage 224 and are executable to provide the functionality of the computing device 220 described herein.

The input interface 230 is used to enter data or commands and can include, for example, a keyboard, a user pointing device such as, for example, a mouse, a trackball, or a touch pad, or may further include the touchscreen or microphone.

The output interface 232 outputs information for reporting or storage in the data storage 224, and thus, the output interface 232 may be similar to the communication interface 228 and can be a wireless interface (e.g., transmitter) or a wired interface as well.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples describe different advantages as compared to other advantageous examples. The example or examples selected are chosen and described to explain the principles of the examples, the practical application, and to enable others of ordinary skill in the art to understand the disclosure for various examples with various modifications as are suited to the particular use contemplated.

Having described the subject matter of the present disclosure in detail and by reference to specific examples thereof, it is noted that the various details disclosed herein should not be taken to imply that these details relate to elements that are essential components of the various examples described herein, even in cases where a particular element is illustrated in each of the drawings that accompany the present description. Further, it will be apparent that modifications and variations are possible without departing from the scope of the present disclosure, including, but not limited to, examples defined in the appended claims. More specifically, although some aspects of the present disclosure are identified herein as preferred or particularly advantageous, it is contemplated that the present disclosure is not necessarily limited to these aspects.

Moreover, while some examples have been described in the context of fully functioning computers and computer systems, those skilled in the art will appreciate that various examples are capable of being distributed as a program product in a variety of forms, and that the disclosure applies equally regardless of a particular type of machine or computer-readable media used to effect the distribution.

Further examples of machine-readable storage media, machine-readable media, or computer-readable (storage) media include, but are not limited to, recordable type media such as volatile and non-volatile memory devices, floppy and other removable drives, hard drives, optical disks (e.g., Compact Disk Read-Only Memory (CD ROMS), Digital Versatile Disks, (DVDs), etc.), among others, and transmission type media such as digital and analog communication links.

For the purposes of describing and defining examples herein, it is noted that terms “substantially” or “about” are utilized herein to represent an inherent degree of uncertainty attributed to any quantitative comparison, value, measurement, or other representation. The terms “substantially” and “about,” when utilized herein, represent the degree by which a quantitative representation may vary from a stated reference without resulting in a change in a basic function of the subject matter at issue, such as varying by 0-2% of the quantitative measurement.

What is claimed is:

1. A system for creating a reactive media to capture a constituent gas from a gas mixture, the system comprising:
a housing into which a non-reactive media is positioned, wherein the housing includes a housing outlet to vent the housing;
a heating element for heating the non-reactive media in the housing which causes a release of gas and creates a reactive media, wherein the gas is directed through the housing outlet;
an emissions measuring device, including a plurality of sensors to detect concentrations of gas constituents and a gas flow sensor to detect a gas flow rate, coupled to the housing outlet; and
a computing device having one or more processors, to perform functions comprising:
calculating an emission of a particular gas constituent in the gas directed through the housing outlet based on a concentration of the particular gas constituent detected by the plurality of sensors and the gas flow rate detected by the gas flow sensor.

2. The system of claim 1, wherein the reactive media is an oxide or a hydroxide.

3. The system of claim 1, wherein:
the non-reactive media is a carbonate input; and
heating the carbonate input causes a release of carbon dioxide and creates a metal oxide, wherein the carbon dioxide is directed through the housing outlet.

4. The system of claim 1, wherein the housing is a sealed housing.

5. The system of claim 1, further comprising:
a thermal recycling system within the housing for receiving and maintaining heat from the heating element and for broadcasting maintained heat in the housing enabling non-continuous operation of the heating element while still maintaining a desired temperature inside the housing capable of creating the reactive media.

6. The system of claim 1, wherein the heating element is powered by a source of power including electric power, hydroelectric power, nuclear power, or a renewable power source.

7. The system of claim 1, wherein the heating element includes resistive heating elements of an electric kiln or rotary furnace.

8. The system of claim 1, wherein the heating element uses one or more of direct microwave heating, thermal focusing, heating from regeneration of a secondary process, and heating through plasma processes.

9. The system of claim 1, wherein the heating element includes a control processor to control operation of the heating element, through use of a source of power, based on a temporal or weather based parameter including a time of day.

10. The system of claim 1, wherein the computing device further performs functions comprising:
determining an amount of power utilized by the heating element that is based on a source of power including a fossil fuel; and
calculating a carbon footprint metric of the reactive media based on (i) the amount of power utilized by the heating element that is based on the source of power including the fossil fuel, (ii) an amount of the emission of the particular gas constituent released during creation of the reactive media, and (iii) an amount of the constituent gas that the reactive media is capable of capturing.

11. The system of claim 1, wherein the computing device further performs functions comprising:
determining a power source carbon footprint based on power utilized by the heating element from a source of power; and
calculating a carbon footprint metric of the reactive media based on (i) the power source carbon footprint, (ii) an amount of the emission of the particular gas constituent released during creation of the reactive media, and (iii) an amount of the constituent gas that the reactive media is capable of capturing.

12. The system of claim 1, wherein the computing device further performs functions comprising:
receiving an input indicating an amount of the reactive media created;
determining, based on the amount of the reactive media created, an amount of the constituent gas that the reactive media is capable of capturing; and
calculating a carbon footprint metric of the reactive media based on an amount of the emission of the particular gas constituent released during creation of the reactive media and an amount of the constituent gas that the reactive media is capable of capturing.

13. The system of claim 12, wherein the computing device further performs functions comprising:
based on the carbon footprint metric being above a threshold, changing a source of power by which the heating element is powered.

14. The system of claim 1, further comprising:
a gas capture device coupled to the housing outlet to receive the gas directed from the housing outlet and to capture the gas directed from the housing outlet.

15. The system of claim 14, wherein the gas capture device compresses captured gas for storage in a container.

16. A system for creating a reactive media to a constituent gas from a gas mixture, the system comprising:
a housing into which a non-reactive media is positioned, wherein the housing includes a housing outlet to vent the housing;
a heating element for heating the non-reactive media in the housing which causes a release of gas and creates a reactive media, wherein the gas is directed through the housing outlet;
a gas capture device coupled to the housing outlet to receive the gas directed from the housing and to capture the gas directed from the housing;
an emissions measuring device, including a plurality of sensors to detect concentrations of gas constituents and a gas flow sensor to detect a gas flow rate, coupled to the housing outlet; and
a computing device having one or more processors, to perform functions comprising:
calculating an emission of a particular gas constituent in the gas directed through the housing outlet based on a concentration of the particular gas constituent detected by the plurality of sensors and the gas flow rate detected by the gas flow sensor; and
calculating a carbon footprint metric of the reactive media based on an amount of the emission of the particular gas constituent released during creation of the reactive media and an amount of the constituent gas that the reactive media is capable of capturing.

17. The system of claim 16, wherein the heating element includes a control processor to control operation of the heating element, through use of a source of power, based on a temporal or weather based parameter including a time of day.

18. The system of claim 16, wherein the computing device further performs functions comprising:
receiving an input indicating an amount of the reactive media created;
determining, based on the amount of the reactive media created, the amount of the constituent gas that the reactive media is capable of capturing.

19. The system of claim 16, wherein the computing device further performs functions comprising:
determining a power source carbon footprint based on power utilized by the heating element from the power source; and
calculating the carbon footprint metric of the reactive media based on (i) the power source carbon footprint, (ii) the amount of the emission of the particular gas constituent released during creation of the reactive media, and (iii) the amount of the constituent gas that the reactive media is capable of capturing.

20. A method for creating a reactive media to a constituent gas from a gas mixture, the method comprising:
heating a non-reactive media positioned in a housing which causes a release of gas and creates a reactive media, wherein the gas is directed through a housing outlet;
detecting, by a plurality of sensors, concentrations of gas constituents in the gas directed through the housing outlet;
detecting, by a gas flow sensor, a gas flow rate of the gas directed through the housing outlet; and
calculating, by a computing device having one or more processors, an emission of a particular gas constituent in the gas directed through the housing outlet based on a concentration of the particular gas constituent detected by the plurality of sensors and the gas flow rate detected by the gas flow sensor.

21. The method of claim 20, further comprising:
determining an amount of power utilized during the heating that is based on a source of power including a fossil fuel; and
calculating, by the computing device, a carbon footprint metric of the reactive media based on (i) the amount of power utilized by heating that is based on the source of power including the fossil fuel, (ii) an amount of the emission of the particular gas constituent released during creation of the reactive media, and (iii) an amount of the constituent gas that the reactive media is capable of capturing.

22. The method of claim 20, further comprising:
receiving an input indicating an amount of the reactive media created;
determining, based on the amount of the reactive media created, an amount of the constituent gas that the reactive media is capable of capturing; and
calculating, by the computing device, a carbon footprint metric of the reactive media based on an amount of the emission of the particular gas constituent released during creation of the reactive media and an amount of the constituent gas that the reactive media is capable of capturing.
